(19) United States
(12) Patent Application Publication
(10) Pub. No.: US 2002/0032426 A1
(43) Pub. Date: Mar. 14, 2002

(54) Absorbent article

(76) Inventors: Asa Lindstrom, Goteborg (SE); Christina Ragnarsson, Goteborg (SE); Kent Hermansson, Vastra Frolunda (SE)

Correspondence Address: Ronald L. Grudziecki, BURNS, DOANE, SWECKER & MATHIS, L.L.P., P.O. Box 1404, Alexandria, VA, 22313-1404, US

(21) Appl. No.: 09/950,686

(22) Filed: Sep. 13, 2001

Related U.S. Application Data
(60) Provisional application No. 60/232,138, filed on Sep. 13, 2000.

(30) Foreign Application Priority Data
Sep. 13, 2000 (SE) 0003252-4

(51) Int. Cl. A61F 13/15; A61F 13/20

(52) U.S. Cl. 604/385.01; 604/389; 604/391; 604/392

(57) ABSTRACT

Absorbent article such as a diaper and an incontinence guard comprising a pair of belt portions (9) attached to the rear portion (6) alternatively the front portion (5) of the article and which are intended to be fastened together around the waist of the wearer and where said front portion (5) alternatively the rear portion (6) is provided with fastening means (8) intended to be attached to the belt portions (9), in such a way that the article will assume a pantlike shape, where the belt portions (9) form a part of the waist portions of the pant. Said first fastening means (8) at least include an adhering surface (12, 12′) and surface of mechanical fastening means, especially hook and loop fasteners (11), whereby at least the adhering surface (12, 12′) is arranged on a unfoldable tab (15), which in a first folded position covers the mechanical fastening means (11) and in a second unfolded position uncovers this.

### TECHNICAL FIELD

The present invention refers to an absorbent article such as a diaper and an incontinence guard comprising a liquid permeable topsheet, a liquid impermeable backsheet and an absorbent body enclosed therebetween, said article having a front portion, a rear portion and a crotch portion therebetween, and further is provided with a pair of belt portions attached to the rear portion alternatively the front portion of the article and which are intended to be fastened together around the waist of the wearer and where said front portion alternatively said rear portion is provided with attachment means intended to be attached to the belt portions, in such a way that the article will assume a pantlike shape, where the belt portions form a part of the waist portions of the pant.

### BACKGROUND OF THE INVENTION

Diapers and incontinence guards for incontinent adults usually have a garment portion holding an absorbent body in place against the user's body and attachment means which hold the garment portion in place also when the user is moving. A common type of attachment means are adhesive tapes or hook and loop fasteners of the touch-and-close type which directly attach the front and rear portions of the absorbent article to each other. It is further known, through, e.g., EP-A-0 287 388, EP-A-0 409 307, EP-A-0 528 282, EPA-0 605 012 and FR-A-2 586 558, to attach the front and rear portions of the article by means of a belt, at which the possibilities to adjust the fit are improved. The belt further provides a simplified change of diaper or incontinence guard, especially when the patient is standing up.

On a common type of belt diaper the belt portions are first attached around the waist on the patient and then the front portion of the diaper is attached to the outside of the belt using hook and loop fasteners, being arranged at the front portion and being attached against the belt portions, which are of nonwoven material and thereby may function as a reception surface for hook and loop fasteners. One problem with diapers provided with hook and loop fasteners and a plastic backsheet material, is that on smaller persons the strap having the hook and loop fasteners on the front portion extends beyond the reception surface for the hook and loop fastener on the belt portions, all the way to the plastic backsheet on the rear portion of the diaper. The hook and loop fasteners cannot attach to this plastic material. If in this case the front portion is anyhow attached to the belt, this will lead to a poor fit. It would therefore be desirable to be able to provide a diaper or incontinence guard which will fit persons of different sizes.

It is previously known with combined fastening means having hook and loop fasteners and tape tabs, respectively. These have principally been present to be able to form a package of the used diaper for disposal in the wastes. See, e.g., WO 98/27922, U.S. Pat. No. 5,019,065 and U.S. Pat. No. 4,869,724. There are also combined fastening means having a inner tape surface and an outer hook and loop fastener in order to protect the fastening means at transport before usage or to get soiled from, e.g., baby oil, powder, etc., before the final application. See WO 97/28774 and WO 93/19712, respectively. None of the documents shows a combined fastening means which improves the fit of the diaper.

### SUMMARY OF THE INVENTION

The object of the present invention is to accomplish a belt-provided diaper or incontinence guard having improved possibilities to adjust the fit to be able to adapt to persons having different waist sizes. This object is being solved in that the fastening means which is arranged at the front portion of the diaper at least include one adhering surface and a surface having mechanical fastening means, especially hook and loop type of fasteners, wherein at least the adhering surface is arranged on a unfoldable tab, which in a first folded position covers the mechanical fastening means and in a second unfolded position uncovers this. In the case of a small person, one may have to attach the fastening means of the front portion against the rear portion, which usually exhibits a plastic film on the outside. The adhering surface of the fastening means may hereby be used. However, for a normally sized or large sized person the mechanical fastening means are used which attach directly against the belt portions. A good fit of the product is hereby provided on a small person as well as on a normally sized or large sized person. Said adhering surface includes adhesive, tape tabs or the like.

### BRIEF DESCRIPTION OF DRAWINGS

The invention will in the following be closer described with reference to embodiments shown in the accompanying drawings.

FIG. 1 shows schematically a perspective view of a diaper or incontinence guard according to the invention having the fastening means on the front portion in a folded and in a unfolded state.

FIG. 2 shows an alternative embodiment of the fastening means on the front portion on a diaper according to FIG. 1.

### DESCRIPTION OF AN EMBODIMENT

The drawing shows an embodiment of a diaper or incontinence guard 1 comprising a liquid impermeable backsheet 2, a liquid permeable topsheet 3 and an absorbent body 4 enclosed therebetween. The liquid permeable topsheet 3 can include a nonwoven material, e.g., a spunbond material of continuous filaments, a meltblown material, a bonded carded fibrous web or a perforated plastic film. The liquid impermeable backsheet 2 may include a plastic film, a nonwoven material coated with a liquid impervious material or a hydrophobic nonwoven material which resists liquid penetration. In the present invention the backsheet material includes a plastic film, which cannot function as a reception surface for the hook and loop fasteners. The topsheet 3 and the backsheet material 2 have a somewhat greater extension in the plane than the absorbent body 4 and extend outside the edges thereof. The layers 2 and 3 are connected to each other within the projecting portions thereof, e.g., by gluing or welding by heat or ultrasonic.

The absorbent body 4 can be of any conventional kind. Examples of commonly occurring absorbent materials are cellulosic fluff pulp, tissue layers, highly absorbent polymers (so called superabsorbents), absorbent foam materials, absorbent nonwovens or the like. It is common to combine cellulosic fluff pulp with superabsorbents in an absorbent body. It is also common to have absorbent bodies comprising layers of different material with different properties with respect to liquid acquisition capacity, liquid distribution capacity and storage capacity. It is well-known to the person skilled in the art and does therefore not have to be described in detail. The thin absorbent bodies which are common in for example baby diapers and incontinence guards often comprise a compressed mixed or layered structure of cellulosic fluff pulp and superabsorbent.

The diaper/incontinence guard is intended to enclose the lower part of the wearer's trunk like a pair of absorbent pants. It comprises a front portion 5 intended during use to be worn on the front part of the user's body, a rear portion 6 intended during use to be worn on the rear part of the user's body, and a more narrow crotch portion 7 located between the front and rear portions and which is intended to be worn in the crotch part of the user between the legs.

A pair of belt portions 9 are with one end attached, e.g., glued or ultrasonically welded, to the rear portion 6 of the diaper. The belt portions 9 are with their opposite ends intended to be fastened together, e.g., by means of fastening means 10 which is attached against the outside of the opposite belt portion. The fastening means 10 may be hook and loop type fasteners or tape tabs. The fastening means 8 of the front portion 5 are intended to be attached against the outsides of the belt portions 9 in order to fasten together the diaper/incontinence guard to the desired pantlike shape.

According to an alternative embodiment the belt portions are attached to the front portion 5 of the diaper and thus are intended to be fastened together on the back of the wearer. The fastening means 8 are then arranged on the rear portion 6 of the diaper.

The width of the belt portions 9 should be between 5-20 cm, preferably between 7-15 cm. The belt portions 9 are preferably a laminate of a carrier material, which forms the outside of the belt and a soft nonwoven, which forms the inside of the belt intended to be in direct contact with the skin of the user. A suitable nonwoven material can be a spunbond material of, e.g., polypropylene- or polyethylene fibres. Conjugate fibres may also be used. Another suitable nonwoven material can be a carded thermobonded material of, e.g., polypropylene-, polyester- or conjugate fibres. Nonwoven or another suitable material may for instance be used as carrier material. The carrier material should be adapted to function as a reception surface for both the attachment means 8 and 10. Also elastic laminates are suitable to use as material in the belt portions.

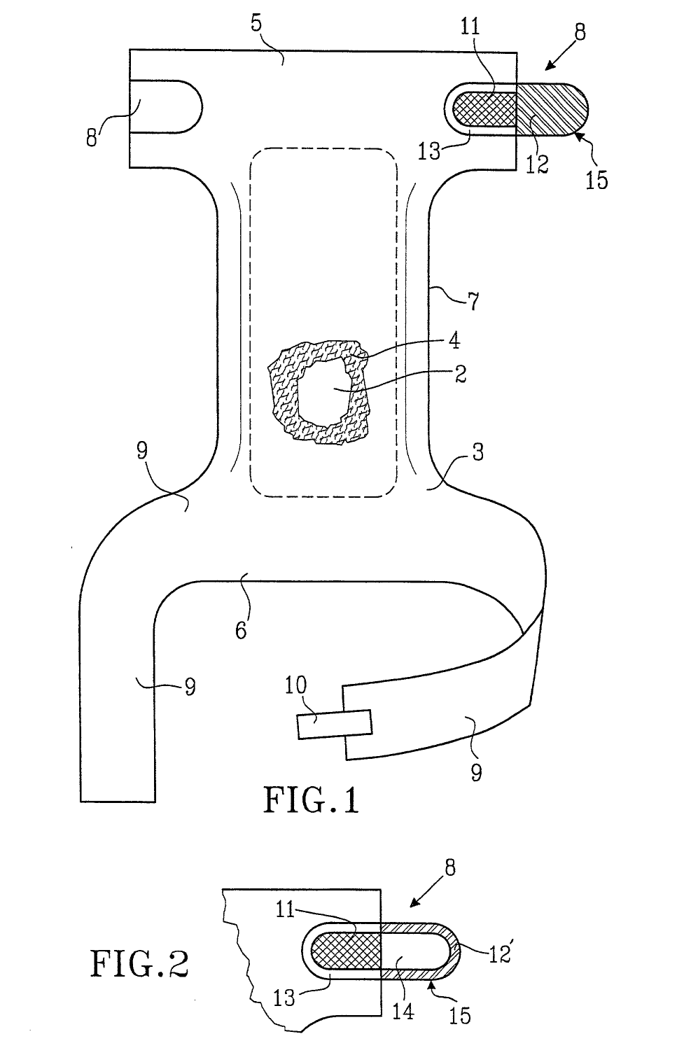

FIG. 1 show the fastening means 8 according the present invention located on the front portion 5 for attachment against the belt. Said fastening means 8 at least include an adhering surface 12 and a surface having mechanical fastening means in the form of hook material 11. The hook material surface 11 comprises one part of a hook and loop type fastener, having the capability to attach to the nonwoven outside of the belt portions 9, which functions as loop material to the hook material 11. The adhering surface 12 may comprise an adhesive surface, a tape tab or the like. The adhering surface 12 is arranged on a unfoldable tab 15 which in a first folded position covers the hook material 11 and in a second folded position uncovers this. When the product is applied on a small person the tab 15 having the hook material extend beyond the loop material reception surface on the belt portions 9 all the way to the backsheet material 2. The backsheet material 2 comprises of plastic material where the hook material 11 cannot attach. In this case said adhering surface 12 may be used to attach the front portion 5 against the plastic backsheet material 2 to achieve the pantlike structure. However, when the product is applied on a normally sized or a large sized person the hook material 11 of the fastening means 8 is used to attach against the belt portions 9.

FIG. 1 shows that said fastening means 8 also includes a second surface 13, which is surrounding the hook material surface 11. Before usage the adhering surface 12 is unfolded from the hook material surface 11 and said second surface 13. Said second surface is comprised of a so-called release material having the capability to easily release the adhering surface 12 making it simple to grip the tab having the adhering surface 12 for application.

In another embodiment the adhering surface 12′ is preferably only arranged along the edge of the tab 15, (see FIG. 2). The area of the adhering surface 12′ then corresponds to the area of said second surface having release material. The central portion 14 of the tab includes a material which will not adhere to the hook material. This design protects the hook material surface 11 from adhering material such as adhesive, when the fastening means is in the folded state.

The diaper may now be adapted to different persons depending on their size with remained fit. It leads to a higher flexibility in, for instance, hospitals and in elderly care, when the same diaper size may be used to an increased number of persons.

The invention is of course not limited to the above described embodiments but can be modified within the scope of the claims.

1. Absorbent article such as a diaper and an incontinence guard comprising a liquid permeable topsheet, a liquid impermeable backsheet and an absorbent body enclosed therebetween, said article having a front portion, a rear portion and a crotch portion therebetween, and further is provided with a pair of belt portions attached to the rear portion alternatively the front portion of the article and which are intended to be fastened together around the waist of the wearer and where said front portion alternatively the rear portion is provided with fastening means intended to be attached to the belt portions, in such a way that the article will assume a pantlike shape, where the belt portions form a part of the waist portions of the pant, wherein said fastening means at least include an adhering surface and a surface of mechanical fastening means, especially hook and loop fasteners, whereby at least the adhering surface is arranged on a unfoldable tab, which in a first folded position covers the mechanical fastening means and in a second unfolded position uncovers this.

2. Absorbent article according to claim 1, wherein the mechanical fastening means is arranged on the inside of the front portion of the article in connection to its side edge.

3. Absorbent article according to claim 1, wherein the adhering surface is arranged along the outer edge on said tab whilst the central portion of this, which in the folded position of the tab is intended to lie close to the mechanical fastening means, is free from adhering material.

4. Absorbent article according to claim 2, wherein the adhering surface is arranged along the outer edge on said tab whilst the central portion of this, which in the folded position of the tab is intended to lie close to the mechanical fastening means, is free from adhering material.

5. Absorbent article according to claim 3, wherein the outer edge of the mechanical fastening means, a portion is arranged with a so called release material intended to serve as a temporary fastening surface for the adhering surfaces of the fastening means in the folded position of the tab.
